(19) United States
(12) Patent Application Publication
(10) Pub. No.: US 2026/0146242 A1
(43) Pub. Date: May 28, 2026

(54) ALPHA-GALACTOSIDASE PROTEIN FOR ENZYME REPLACEMENT THERAPY (ERT) AND METHODS OF USE

(71) Applicant: The Medical College of Wisconsin, Inc., Milwaukee, WI (US)

(72) Inventors: Jeffrey A. Medin, Shorewood, WI (US); Simone Scalia, Milwaukee, WI (US)

(21) Appl. No.: 19/461,195

(22) Filed: Jan. 27, 2026

Related U.S. Application Data
(62) Division of application No. 17/640,246, now Pat. No. 12,565,643.
(60) Provisional application No. 62/902,211, filed on Sep. 18, 2019.

(51) Int. Cl. C12N 9/40; A61K 38/00; A61K 48/00

(52) U.S. Cl. CPC C12N 9/2465; C12Y 302/01022; A61K 38/00; A61K 48/00

(57) ABSTRACT

The present disclosure provides less immunogenic mutant α-galactosidase A protein (α-gal), methods of making, and methods of use. The less immunogenic mutant α-gal of the present invention provides a reduced immune response when administered to a subject. Methods of treating Fabry in a subject are also provided, the methods comprising administering the disclosed less immunogenic mutant α-gal.

### CROSS-REFERENCE TO RELATED APPLICATIONS

This patent application is a divisional of U.S. patent application Ser. No. 17/640,246, filed Mar. 3, 2022, which is the U.S. National Stage entry of PCT/US2020/051570, filed Sep. 18, 2020, which claims the benefit of priority of U.S. Provisional Patent Application No. 62/902,211, filed Sep. 18, 2019, each of which are incorporated herein by reference in their entirety.

### STATEMENT REGARDING FEDERALLY SPONSORED RESEARCH

N/A

### SEQUENCE LISTING

A Sequence Listing accompanies this application and is submitted as an ST26 format file of the sequence listing named “650053_01269.xml” which is 20,970 bytes in size and was created on Jan. 27, 2026. The sequence listing is electronically submitted via Patent Center with the application and is incorporated herein by reference in its entirety.

### BACKGROUND OF THE INVENTION

The field of the invention is mutated alpha galactosidase with reduced immunogenicity for use in the treatment of Fabry disease.

Fabry disease (FD) is a lysosomal storage disease (LSD) resulting from a deficiency in the enzyme α-galactosidase A (α-gal A encoded by the AGA transgene), an enzyme that hydrolyses α-galactose from glycosphingolipids, in particular globotriaosylceramide (Gb₃). Lysosomal Storage Disorders (LSD) are a group of more than 70 rare inherited metabolic disorders that result from lysosome dysfunction, usually as a consequence of a deficiency in a single enzyme required for the intracellular digestion of lipids, glycoproteins or polysaccharides. Because of such deficiencies, the molecules that would normally be degraded accumulate inside the cell, leading to dysfunction or death of the cell.

The standard-of-care treatment for Fabry disease is enzyme replacement therapy (ERT. aka ET). The efficacy of ERT is outlined by Rombach et al. (Orphanet J Rare Dis. 8:47-10.1186/1750-1172-8-47 (2013)). While some benefits can be obtained, disease progression is not halted. ERT requires lengthy intravenous infusions of α-gal administered every couple of weeks, often at an outpatient center. Although Fabry disease is relatively rare, there are about 4000 patients in the US, treatment costs are on the order of $300,000/year/patient ($1.2 B/year for all US patients).

Fabry patients sometimes have an immune reaction to the ERT. Therefore, there is a need for less immune reactive α-gal A that can be used for enzyme replacement therapy.

### SUMMARY OF THE INVENTION

The present invention overcomes the aforementioned drawbacks by providing mutant α-galactosidase A proteins that are less immunogenic, nucleotides encoding the mutant α-galactosidase A proteins, and methods of making and using the same.

In one aspect, the present invention provides less immunogenic mutant α-galactosidase A protein (α-gal) comprising one or more amino acid changes in one or more antigenic epitopes, wherein the one or more antigenic epitopes are selected from the group consisting of (a) LHWERF (SEQ ID NO:2; position 45-50 of α-gal), (b) DCQEEPDSCI (SEQ ID NO: 3; position 55-64 of α-gal), (c) FMEMAELMVSEQ (SEQ ID NO:4; position 69-79 of α-gal), (d) KDAGYEY (SEQ ID NO:5; position 82-88 of α-gal), (e) DDCWMA (SEQ ID NO:6; position 92-97 of α-gal), (f) EGRLQADPQRF (SEQ ID NO:7; position 103-113 of α-gal), (g) TCAGF (SEQ ID NO:8; position 141-145 of α-gal), (h) DIDAQTF (SEQ ID NO:9; position 153-159 of α-gal), (i) DLLKFDGC (SEQ ID NO:10; position 165-172 of α-gal), (j) ENLAD (SEQ ID NO:11; position 178-182 of α-gal), (k) IVYSCEW (SEQ ID NO:12; position 198-204 of α-gal), (l) PLYMWPFQ (SEQ ID NO:13; position 205-212 of α-gal), (m) YCNHW (SEQ ID NO:14; position 222-226 of α-gal), (n) SWKSI (SEQ ID NO:15; position 235-239 of α-gal), (o) LDWTSFNQER (SEQ ID NO:16; position 243-252 of α-gal), (p) IVDVA (SEQ ID NO:17; position 253-257 of α-gal), (q) NDPDML (SEQ ID NO: 18; position 263-268 of α-gal), (r) ALLQD (SEQ ID NO: 19; position 309-313 of α-gal), (s) QLRQGDNF (SEQ ID NO:20; position 330-337 of α-gal), (t) EVWERPLSG (SEQ ID NO:21; position 338-346 of α-gal), (u) WAVAMIN (SEQ ID NO: 22; position 349-355 of α-gal), and (v) EIGGPRSY (SEQ ID NO:23; position 358-365 of α-gal), wherein the mutant α-gal retains enzymatic function and is less immunogenic than the non-mutated α-gal.

In another aspect, an expression construct comprising a nucleic acid sequence encoding the mutant α-gal described herein is contemplated.

In another aspect, the disclosure provides a cell able to express the mutant α-gal described herein. In one example, the cell is a patient's cell. In another example, the patient cell is used for therapy.

In another aspect, the disclosure provides a method of producing a mutant α-gal, the method comprising: a) expressing the mutant α-gal in a cell; and (b) isolating and purifying the mutant α-gal protein from the cell, wherein the mutant α-gal has enzymatic activity.

In another aspect, the disclosure provides a method of treating Fabry disease in a subject, the method comprising the steps of: (a) administering a therapeutically effective amount of the disclosed mutant α-gal proteins to treat Fabry disease and reduce one or more symptoms of the lysosomal storage disorder.

The foregoing and other aspects and advantages of the invention will appear from the following description. In the description, reference is made to the accompanying drawings which form a part hereof, and in which there are shown, by way of illustration, preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

FIG. 1A. Sera from 58 male and female FD patients were analyzed for the presence of anti-α-gal A IgG by ELISA. High levels (>5-fold greater than healthy donors) of antibody against α-gal A were found in 21 patients (36%). Male patients showed a statistically significant higher level of anti-α-gal A IgG in comparison to females. Dashed line at 2 indicates cut-off for low-titer patients. * p<0.05, Mann-Whitney U test.

FIG. 1B. Anti α-gal A IgG levels as measured by ELISA in Fabry patients treated with Fabrazyme, Replagal, both enzyme therapies (switch), or no enzyme therapy. Male Fabry patients display higher Anti α-gal A IgG than female Fabry patients. Both Fabrazyme and Replagal treated Fabry patients both express anti-α-gal A IgG. Enzyme Therapy naïve Fabry patients also display anti-α-gal A IgG.

FIG. 2. Epitope mapping analysis was performed on the sera of 17 high-titer and 20 low-titer FD patients. Whereas 5 reactive epitopes of α-gal A were identified in high-titer patients, only a single epitope (MKE3) was present in low-titer patients.

FIG. 3. Patient epitope mapping results were grouped according to their ERT therapy. Sera from aga β-treated patients contained all 5 of the previously identified epitopes while sera from aga α-treated patients was missing epitope MKE5.

FIG. 4. An anti-gal A immune response generated in Fabry mice was evaluated by ELISA. PEPperMAP confirmed that the epitope profile detected was similar to that of the high-titer patients (3 of the 5 identified epitopes were present).

FIG. 5. IgG epitope mapping revealed many sites within the α-gal A protein that were immunogenic. Several sites were identified as being immunogenic across male Fabry patients, female Fabry patients, and Fabry mice.

FIG. 6. Novel α-gal A variants were selected using software to identify mutations that are predicted to maintain enzyme functionality (software used: PolyPhen-2) while reducing immunogenicity (databases used: CTLpred, Immunomedicine group, MHCBP, IEDB.org, SVRMHC, BcePred, SVMTrip, PREDIVAC, EpiJen). Notably, these mutations are found in three of the five reactive epitopes identified in Example 1. In many cases there are multiple amino acid changes possible per mutation predicted to retain functionality while reducing immunogenicity. Red boxes are used to highlight the six mutations that were selected for in vitro analysis in Example 3.

FIG. 7A. Enzyme activity assays were performed on media collected from cultured cells expressing the six indicated α-gal A variants. Enzyme activity is expressed as a percentage of wild-type activity. Three of the 7 mutant secreted proteins retained near wild-type enzymatic activity.

FIG. 7B. Enzyme activity assays were performed on the lysate of cells expressing the six indicated α-gal A variants. Enzyme activity is expressed as a percentage of wild-type activity. Intracelluar enzyme activity retention was similar to that of secreted in FIG. 7A.

FIG. 8. Protein expression of the α-gal A variants was confirmed using western blot analysis with an anti-α-gal A antibody. Expression correlated with the level of enzyme activity in FIGS. 7A and 7B.

FIG. 9. mRNA expression of the α-gal A variants was confirmed using quantitative reverse transcription PCR. The results are shown as a percentage of wild-type expression. Transcript for each of the variant proteins was either equal to or above the level of transcript for the wild-type α-gal A.

### DETAILED DESCRIPTION OF THE INVENTION

The present disclosure provides improved mutant alpha galactosidase A protein (α-gal A) for use in treating Fabry disease (FD). Particularly, the present invention describes a mutant α-gal A protein that has reduced immunogenicity in a subject while maintaining its enzymatic function, and methods of using the mutant α-gal to deliver therapeutic products to a subject in need thereof.

FD is an α-gal A deficiency in which globotriaosylceramide (Gb₃) and other metabolites accumulate. Standard of care for Fabry patients has been enzyme replacement therapy (ERT), e.g., regular treatment with exogenous α-galactosidase A. For example, in the United States there is a commercially approved Enzyme Replacement Therapy (ERT) to treat Fabry disease called agalsidase beta (agalsidase-β, Fabrazyme®) produced by Sanofi-Genzyme (produced in Chinese hamster ovary cells). It is administered by intravenous infusion usually every two weeks. The approved dose is 1 milligrams per kilogram of body weight. In Europe and many other countries but not in the United States, there is also a commercially approved intravenous Enzyme Replacement Therapy (ERT) to treat Fabry disease called agalsidase alfa (agalsidase-α, Replagal®) produced by Shire (produced from a modified human fibrosarcoma cell line). It is also administered by intravenous infusion usually every two weeks. The approved dose is 0.2 milligrams per kilogram of body weight. Two new plant-based products are currently in development: pegunigalsidase α, (Protalix Biotherapeutics), which is produced in BY2 tobacco cells, and moss-αGal (Greenovation), which is produced in moss.

The immune responses, including humoral immune responses following enzyme replacement therapy have been described in the literature for Fabry disease.

The mutations discovered in the present invention described herein may be useful in the reduction of the immune response for the ERTs already on the market. By incorporating one or more of the mutations described herein in turn reduces the immune response toward the exogenous α-gal and one or more of the associated side effects. The ability to reduce the immune response to the exogenously derived α-gal in turn increases the efficacy of the ERT. Not to be bound by theory, the ability to reduce the immunogenicity of the exogenous α-gal A protein reduces the humoral responses to exogenous protein, in effect decreasing the likelihood of producing neutralizing antibodies to the exogenous α-gal protein, and thus increasing the efficacy of ERT by avoiding reduced efficacy caused by neutralizing antibodies to the exogenous protein.

The mutant α-gal A proteins of the present disclosure are provided that reduce the immune reaction to exogenously administered α-gal in a patient with Fabry disease. By “reduced immune reaction” the present mutant α-gal elicit a reduced immune reaction to the exogenously administered α-gal protein in a subject having Fabry disease. The immune reaction includes a humoral (antibody) response to the α-gal protein, which may include neutralizing antidrug antibodies (ADAs) which can attenuate the therapeutic efficacy of ERT in a subset of patients.

For clarity, epitopes of α-gal are described herein using position numbers based on the α-gal protein sequence of SEQ ID NO: 1 (NCBI ref: NP_000160.1). However, the amino acid changes of α-gal mutant described herein can be incorporated into other α-gal known in the art that can be used for ERT, including other altered α-gal proteins which may already have some changes within the protein sequence. One skilled in the art would be able to align the protein sequences and translate the position numbers of the epitopes described herein into other known α-gal proteins. Thus, it is contemplated that changes in one or more epitopes that reduce the immunogenicity can be applied to other protein sequences of α-gal known in the art and are part of the present invention. As discussed more below, the ability to test the immunogenicity is well within one skilled in the art taking into account the teachings of the present invention.

In one embodiment, the present disclosure provides a less immunogenic mutant α-galactosidase A protein (α-gal) comprising one or more amino acid changes in one or more antigenic epitopes, wherein the one or more antigenic epitopes are selected from the group consisting of (a) LHWERF (SEQ ID NO:2; position 45-50 of α-gal), (b) DCQEEPDSCI (SEQ ID NO: 3; position 55-64 of α-gal), (c) FMEMAELMVSEQ (SEQ ID NO:4; position 69-79 of α-gal), (d) KDAGYEY (SEQ ID NO:5; position 82-88 of α-gal), (e) DDCWMA (SEQ ID NO:6; position 92-97 of α-gal), (f) EGRLQADPQRF (SEQ ID NO:7; position 103-113 of α-gal), (g) TCAGF (SEQ ID NO:8; position 141-145 of α-gal), (h) DIDAQTF (SEQ ID NO:9; position 153-159 of α-gal), (i) DLLKFDGC (SEQ ID NO:10; position 165-172 of α-gal), (j) ENLAD (SEQ ID NO:11; position 178-182 of α-gal), (k) IVYSCEW (SEQ ID NO:12; position 198-204 of α-gal), (l) PLYMWPFQ (SEQ ID NO:13; position 205-212 of α-gal), (m) YCNHW (SEQ ID NO:14; position 222-226 of α-gal), (n) SWKSI (SEQ ID NO:15; position 235-239 of α-gal), (o) LDWTSFNQER (SEQ ID NO:16; position 243-252 of α-gal), (p) IVDVA (SEQ ID NO:17; position 253-257 of α-gal), (q) NDPDML (SEQ ID NO: 18; position 263-268 of α-gal), (r) ALLQD (SEQ ID NO: 19; position 309-313 of α-gal), (s) QLRQGDNF (SEQ ID NO:20; position 330-337 of α-gal), (t) EVWERPLSG (SEQ ID NO:21; position 338-346 of α-gal), (u) WAVAMIN (SEQ ID NO: 22; position 349-355 of α-gal), and (v) EIGGPRSY (SEQ ID NO:23; position 358-365 of α-gal), wherein the mutant α-gal retains enzymatic function and is less immunogenic than the wild-type or non-mutated α-gal. These mutations are relative to SEQ ID NO:1. In another example, the disclosure provides a less immunogenic mutant α-gal protein comprising one or more amino acid changes in two or more antigenic epitopes selected from the group consisting of (a)-(v), alternatively three or more antigenic epitopes selected from the group consisting of (a)-(v), alternatively four or more antigenic epitopes selected from the group consisting of (a)-(v), alternatively five or more or more antigenic epitopes selected from the group consisting of (a)-(v), and alternatively comprises two or more amino acid changes within the one or more epitopes relative to SEQ ID NO:1.

In some embodiments, the less immunogenic mutant α-gal comprises one or more, alternatively two or more, alternatively three or more amino acid changes in two or more antigenic epitopes selected from the group consisting of (a)-(v), wherein the mutant α-gal retains enzymatic function and is less immunogenic than the non-mutated α-gal.

In one example, the mutant α-gal comprises changes in three or more antigenic epitopes selected from the group consisting of (a)-(v), wherein the mutant α-gal retains enzymatic function and is less immunogenic than the non-mutated α-gal.

As described in the Examples, the epitopes were identified in male and female Fabry patients that where immunogenic as listed in Table 1. Computer analysis of the epitopes was run to determine amino acid changes that allow for the retention of the enzymatic function but reduce the immunogenicity of the epitope within the α-gal.

In another example, the one or more amino acid changes are found in the one or more of the epitopes selected from the group consisting of (a) LHWERF (SEQ ID NO:2; position 45-50 of α-gal), (b) DCQEEPDSCI (SEQ ID NO:3; position 55-64 of α-gal), (c) FMEMAELMVSEQ (SEQ ID NO:4; position 69-79 of α-gal), (d) KDAGYEY (SEQ ID NO:5; position 82-88 of α-gal), (e) DDCWMA (SEQ ID NO:6; position 92-97 of α-gal), (f) EGRLQADPQRF (SEQ ID NO: 7; position 103-113 of α-gal), (g) TCAGF (SEQ ID NO:8; position 141-145 of α-gal), (h) DIDAQTF (SEQ ID NO:9; position 153-159 of α-gal), (i) DLLKFDGC (SEQ ID NO:10; position 165-172 of α-gal), (j) ENLAD (SEQ ID NO:11; position 178-182 of α-gal), (k) IVYSCEW (SEQ ID NO:12; position 198-204 of α-gal), (l) PLYMWPFQ (SEQ ID NO: 13; position 205-212 of α-gal), (m) YCNHW (SEQ ID NO:14; position 222-226 of α-gal), (n) SWKSI (SEQ ID NO:15; position 235-239 of α-gal), (o) LDWTSFNQER (SEQ ID NO:16; position 243-252 of α-gal), (p) IVDVA (SEQ ID NO:17; position 253-257 of α-gal), (q) NDPDML (SEQ ID NO:18; position 263-268 of α-gal), (r) ALLQD (SEQ ID NO:19; position 309-313 of α-gal), (s) QLRQGDNF (SEQ ID NO:20; position 330-337 of α-gal), (t) EVWERPLSG (SEQ ID NO: 21; position 338-346 of α-gal), (u) WAVAMIN (SEQ ID NO:22; position 349-355 of α-gal), and (v) EIGGPRSY (SEQ ID NO:23; position 358-365 of α-gal), alternatively two or more epitopes selected from (a), (b), (d), (i), (k), (o), (p), (q), (r), (s), (t), and (v); alternatively three or more epitopes selected from the group consisting of (a), (b), (d), (i), (k), (o), (p), (q), (r), (s), (t), and (v); alternatively four or more epitopes selected from the group consisting of (a), (b), (d), (i), (k), (o), (p), (q), (r), (s), (t), and (v), and combinations therein.

In some embodiments one or more amino acids may be altered in the epitope identified in (a)-(v), for example, two or more amino acids may be altered in the same epitope (e.g., an epitope selected from (a)-(v)), alternatively three or more amino acids may be altered in the same epitope, alternatively four or more amino acids may be altered in the same epitope.

One skilled in the art would be able to determine and test if an amino acid change that does not alter the enzymatic function, but reduces immunogenicity of the exogenous α-gal protein when administered to a subject.

Suitable mutations within the epitopes identified include an amino acid change at one or more positions within the α-gal protein, wherein the one or more positions is selected from the group consisting of 58, 59, 61, 62, 66, 70, 78, 79, 83, 84, 86, 100, 101, 309, 318, and 323 (relative to SEQ ID NO:1), wherein the amino acid change at one or more of these positions retains enzymatic function and reduces the immune reaction to exogenous α-gal in a subject.

In another example, the mutant α-gal comprises one or more amino acid change selected from the group consisting of E58N, E59N, D61N, S62R, S62N, S62T, E66P, M70A, M70R, M70N, M70D, M70G, M70H, M70Q, M70I, M70L, M70K, M70P, S78D, S78E, S78V, E79A, E79N, E79D, E79G, D83E, A84I, A84V, Y86L, R100F, D101N, D101E, A309N, A318G, and P323R, in alternative examples, the mutant α-gal comprises two or more of the amino acid changes selected from the group consisting of E58N, E59N, D61N, S62R, S62N, S62T, E66P, M70A, M70R, M70N, M70D, M70G, M70H, M70Q, M70I, M70L, M70K, M70P, S78D, S78E, S78V, E79A, E79N, E79D, E79G, D83E, A84I, A84V, Y86L, R100F, D101N, D101E, A309N, A318G, and P323R, alternatively three or more, alternatively four or more, alternatively five or more, alternatively six or more, alternatively seven or more, alternatively eight or more, alternatively nine or more, alternatively ten or more amino acid changes selected from the group consisting of E58N, E59N, D61N, S62R, S62N, S62T, E66P, M70A, M70R, M70N, M70D, M70G, M70H, M70Q, M70I, M70L, M70K, M70P, S78D, S78E, S78V, E79A, E79N, E79D, E79G, D83E, A84I, A84V, Y86L, R100F, D101N, D101E, A309N, A318G, and P323R. The mutant α-gal containing the one or more amino acid changes have maintained enzymatic function but have reduced the immunogenicity (e.g., reduced the humoral response) to the antibody.

“Percentage of sequence identity” or “sequence similarity” is determined by comparing two optimally aligned sequences over a comparison window, wherein the portion of the polypeptide or polynucleotide sequence in the comparison window may comprise substitutions, or additions or deletions (i.e., gaps) as compared to the reference sequence (which does not comprise substitutions, additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “similarity” of polypeptide or polynucleotide sequences means that a polypeptide or polynucleotide comprises a sequence that has at least 80% sequence identity. Suitable sequence similarity allows for small changes in the protein or transgene that do not affect the function of the protein expressed by the transgene. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using programs such as BLAST using standard parameters. These values can be appropriately adjusted to determine corresponding identity of proteins encoded by two nucleotide sequences by taking into account codon degeneracy, amino acid similarity, reading frame positioning and the like.

Methods of testing immunogenicity and enzymatic function are understood by one skilled in the art in view of this disclosure.

As discussed in the examples, a number of the epitopes found within the human patients were also found within mice treated with the exogenous α-gal. Therefore, one skilled in the art would be able to be use the mouse model to test and determine if reduced immunogenicity, e.g., a reduced humoral response, to the exogenous mutant α-gal. The ability to determine the enzymatic function of the protein is known in the art.

The present disclosure also provides an expression construct comprising a nucleic acid sequence encoding the mutant α-gal described herein. Expression constructs comprise a heterologous promoter and the nucleic acid sequence encoding the mutant α-gal protein which is capable of expressed and purified from a cell. The expression constructs include vectors which are capable of directing the expression of exogenous genes to which they are operatively linked. Such vectors are referred to herein as “recombinant constructs,” “expression constructs,” “recombinant expression vectors” (or simply, “expression vectors” or “vectors”). Suitable vectors are known in the art and contain the necessary elements in order for the gene encoded within the vector to be expressed as a protein in the host cell. The term “vector” refers to a nucleic acid molecule capable of transporting another nucleic acid to which it has been linked. One type of vector is a “plasmid”, which refers to a circular double stranded DNA loop into which additional DNA segments may be ligated, specifically exogenous DNA segments encoding the mutant α-gal protein. Another type of vector is a viral vector, wherein additional DNA segments may be ligated into the viral genome. Certain vectors are capable of autonomous replication in a host cell into which they are introduced. Other vectors can be integrated into the genome of a host cell upon introduction into the host cell, and thereby are replicated along with the host genome (e.g. lentiviral vectors). Moreover, certain vectors are capable of directing the expression of exogenous genes to which they are operatively linked. In general, vectors of utility in recombinant DNA techniques are often in the form of plasmids. In the present specification “vector” include expression vectors, such as viral vectors (e.g., replication defective retroviruses (including lentiviruses), adenoviruses and adeno-associated viruses), which serve equivalent functions.

The vectors are heterogeneous exogenous constructs containing sequences from two or more different sources. Suitable vectors include, but are not limited to, plasmids, expression vectors, lentiviruses (lentiviral vectors), adeno-associated viral vectors (rAAV) among others and includes constructs that are able to express the mutant α-gal. A preferred vector is a lentiviral vector. Suitable methods of making lentiviral vector particles are known in the art to be able to transform cells in order to express the mutant α-gal described herein.

The vectors are not limited and include any vectors including lentiviral vectors capable of expressing the mutant α-gal are contemplated for use in the practice of the current invention. A vector can preferably transduce, transform or infect a cell, thereby causing the cell to express the mutant α-gal proteins encoded by the vector.

In another example, the present disclosure provides a virus comprising the expression construct capable of expressing the mutant α-gal.

In another example, a host cell able to express the mutant α-gal described herein are provided. Suitable host cells that can express the mutant α-gal protein includes any tissue culture cell, plant cell or other cell that is able to express the mutant α-gal to retain enzymatic activity. For example, suitable cell types are known in the art and include, but are not limited to, Chinese hamster ovary (CHO) cells, 293 cells, moss cells, BY2 tobacco cells, skin fibroblasts, stem cells, induced pluripotent stem cells (iPSCs), hematologic cells, T cells, liver cells, among others known in the art. Plant cells are also contemplated.

Further, the mutant α-gal proteins can also be used in methods and systems for gene therapy that are being used for treating Fabry disease. For example, mutant α-gal proteins can be used for gene therapy by incorporating the mutant α-gal into a patient's own or donor cell, particularly stem cells, for example hematopoietic stem cells (CD34+ stem cells), or other cells including T-cells, T-rapa cells, among others. For example, patient CD34+ stem cells can be transduced with the mutant α-gal and reintroduced into the patient. Suitable methods of using the mutant α-gal protein include incorporating into a viral gene therapy system (e.g., retroviral including lentiviral vector gene therapy system, AAV gene therapy system, etc.), for example, using the methods described in U.S. Patent Application Nos. PCT/US2018/036292, PCT/US2019/029639, the contents of which are incorporated by reference in their entireties.

The present disclosure also provides compositions comprising the mutant α-gal protein described herein and a pharmaceutically acceptable carrier. A “pharmaceutically acceptable carrier” means any conventional pharmaceutically acceptable carrier, vehicle, or excipient that is used in the art for production and administration of compositions to a subject. Pharmaceutically acceptable carriers are typically non-toxic, inert, solid or liquid carriers which are physiologically balanced. The pharmaceutically acceptable carriers are carriers that allow for the retention of the activity of the enzyme within the composition and they can be stably stored before administration and are known in the art. The carriers keep the proper folding of the mutant α-gal protein that allows enzymatic function. Typically, phosphate buffered saline or other saline solutions are physiologically acceptable carriers. A pharmaceutically acceptable carrier can be selected on the basis of the selected route of administration and standard pharmaceutical practice. The compounds may be formulated into dosage forms according to standard practices in the field of pharmaceutical preparations. See Alphonso Gennaro, ed., Remington's Pharmaceutical Sciences, 18th Ed., (1990) Mack Publishing Co., Easton, Pa, which is incorporated by reference in its entirety. Suitable dosage forms may comprise, but are not limited to, for example, solutions, parenteral solutions, injectable solutions, troches, suppositories, or suspensions.

For parenteral administration, the active agent may be mixed with a suitable carrier or diluent such as water, an oil (e.g., a vegetable oil), ethanol, saline solution (e.g., phosphate buffer saline or saline), aqueous dextrose (glucose) and related sugar solutions, glycerol, or a glycol such as propylene glycol or polyethylene glycol. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

The pharmaceutical composition is preferably in unit dosage form. In such form the preparation is divided into unit doses containing appropriate quantities of the active component. The unit dosage form can be a packaged preparation, the package containing discrete quantities of preparation, such as packeted vials or ampoules.

In another embodiment, the present disclosure provides a method of producing a mutant α-gal, the method comprising: a) expressing the mutant α-gal described herein in a host cell; and (b) isolating and purifying the mutant α-gal protein from the host cell, wherein the mutant α-gal has enzymatic activity and lower immunogenicity compared to the wild type or non-mutant α-gal.

In another embodiment, the disclosure provides a method of treating Fabry disease in a subject, the method comprising the steps of: (a) administering a therapeutically effective amount of the mutant α-gal described herein to treat Fabry disease.

By “treating” we mean the management and care of the subject for combatting the disease, condition or disorder, particularly Fabry disease. The term treating includes the administration of a compound or composition described herein to reduce, ameliorate, or eliminate one or more symptoms of Fabry disease. For example, treating Fabry disease in a subject includes reducing or eliminating the accumulation of spingolipids/glycolipids, particularly globotriaosylceramides within the subject (e.g., reduce the accumulation within blood vessels, and other tissues and organs within the subject). Treatment thus results in the reduction in one or more symptom associated with the accumulation of the glycolipid within the subject. Symptoms vary depending on the organ affected, but include, but are not limited to, e.g., pain, kidney disease, kidney failure, abnormal thickening of the heart muscle or hypertrophy, abnormal heart rhythm, heart valve thickening, valve leakage, angiokeratomas, anhidrosis, neuropathy, fatigue, cerebrovascular effects leading to an increased risk of stroke, vertigo, verterbrobasilar system tinnitus, nausea, inability to gain weight, diarrhea, among others.

In a preferred example, the mutant α-gal is administered by intravenous transfusion.

Treating also encompasses reducing the immune response to exogenous α-gal in a subject. Reduction in the immune response in a subject includes reducing or eliminating a humoral response to exogenous α-gal (e.g., reduction in amount or number of antibodies against exogenous α-gal), reducing or eliminating neutralizing antibodies to exogenous α-gal, and/or reducing other immune response signaling. It is known that antibodies can neutralize α-gal A protein administered by ERT or gene therapy in at least a couple of ways: 1) they can bind to the circulating product in the plasma and block the normal uptake route into cells through the Mannose-6-Phosphate Receptor; 2) they can also bind to α-gal A and inhibit enzyme trafficking and activity in recipient cells. Thus the use and administrating the mutant α-gal described herein can reduce or inhibit one or more of these results of neutralizing antibodies, e.g., reduce antibody binding to the circulating product in the plasma and block the normal uptake route into cells through the Mannose-6-Phosphate Receptor; and/or reduce antibody binding to α-gal A and reduce the ability to inhibit enzyme trafficking and activity in recipient cells.

By “subject” or “patient” refers to a mammal, particularly a human, in which suffer from a disease able to be treated with exogenous α-gal. In a particular example, the subject is a human suffering from Fabry disease. Particularly, the subject is a male or female human suffering from Fabry disease.

Not to be bound by any theory, but it is advantageous to administer an α-gal A protein that has reduced immunogenicity, including the fact that reduction in the immune response that is associated with ERT will reduce the side effects and may prolong the beneficial outcomes from ERT, which will lower treatment morbidity and cost.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. In places where ranges of values are given, this disclosure explicitly contemplates other combinations of the lower and upper limits of those ranges that are not explicitly recited. For example, recitation of a value between 1 and 10 or between 2 and 9 also contemplates a value between 1 and 9 or between 2 and 10. Ranges identified as being “between” two values are inclusive of the end-point values. For example, recitation of a value between 1 and 10 includes the values 1 and 10.

Aspects of the present disclosure that are described with respect to methods can be utilized in the context of the compositions of matter or kits discussed in this disclosure. Similarly, aspects of the present disclosure that are described with respect to compositions of matter can be utilized in the context of the methods and kits, and aspects of the present disclosure that are described with respect to kits can be utilized in the context of the methods and compositions of matter.

The invention will be more fully understood upon consideration of the following non-limiting examples.

### EXAMPLES

### Example 1: Immunogenic Epitope Analysis of Alpha Galactosidase A

In this Example, the inventors identify immune reactive epitopes within the α-gal protein in ERT-treated FD patients. This information was used to determine amino acid substitutions that can be used to develop novel, less immunoreactive α-gal A variants for use in ERT or gene therapy, as these novel α-gal A variants may have improved clinical efficacy.

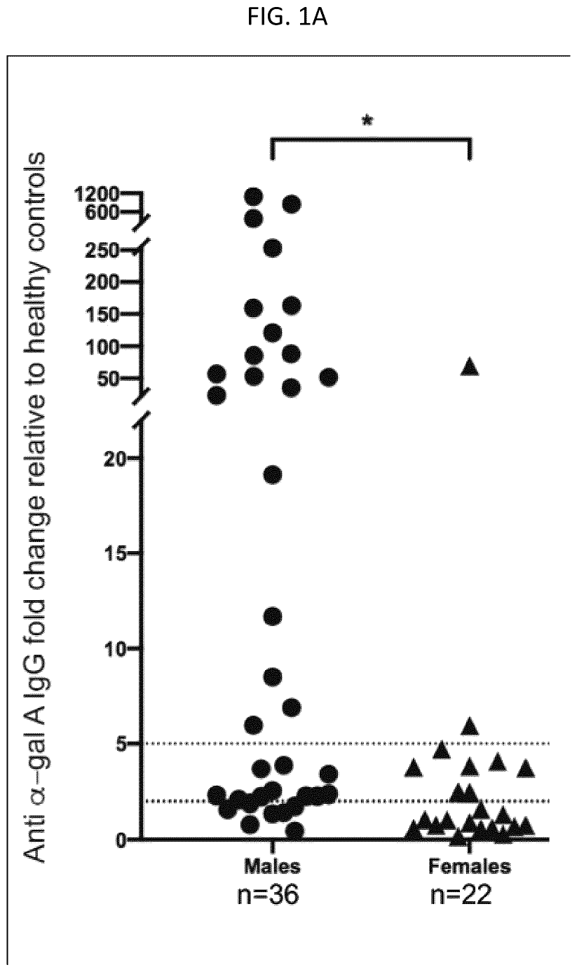

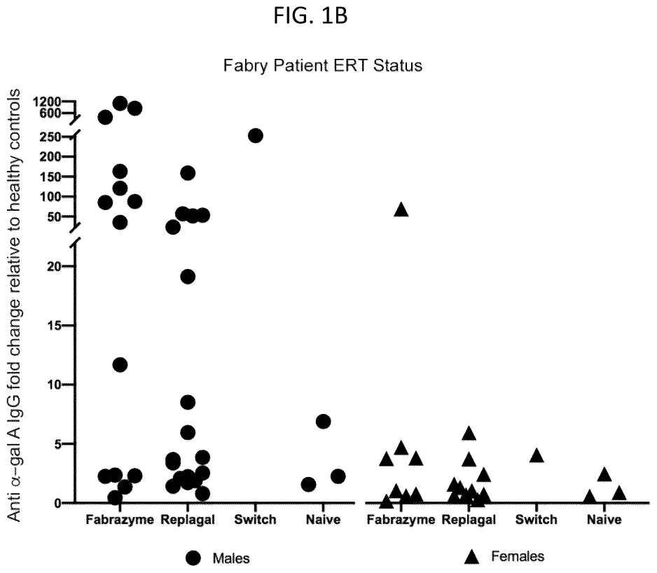

Method: Anti-α-gal A IgG titers were determined via ELISA in 6 non-treated, 28 aga α- and 22 aga β-treated FD subjects. FIG. 1A shows the results of sera from 58 male and female FD patients were analyzed for the presence of anti-α-gal A IgG by ELISA. High levels (>5-fold greater than healthy donors) of antibody against α-gal A were found in 21 patients (36%). Male patients showed a statistically significant higher level of anti-α-gal A IgG in comparison to females.

Also included were 2 FD patients that switched therapy from aga α to aga β. Immunogenic epitopes of α-gal A were identified by PEPperMAP technology (PEPperPRINT GmbH, Hamburg, Germany), as shown in FIG. 1B. FIG. 1B shows anti-α-gal A IgG levels as measured by ELISA in Fabry patients treated with Fabrazyme, Replagal, both enzyme therapies (switch), or no enzyme therapy. Male Fabry patients display higher anti-α-gal A IgG than female Fabry patients. Both Fabrazyme and Replagal treated Fabry patients both express anti-α-gal A IgG. Enzyme therapy naïve Fabry patients also display anti-α-gal A IgG.

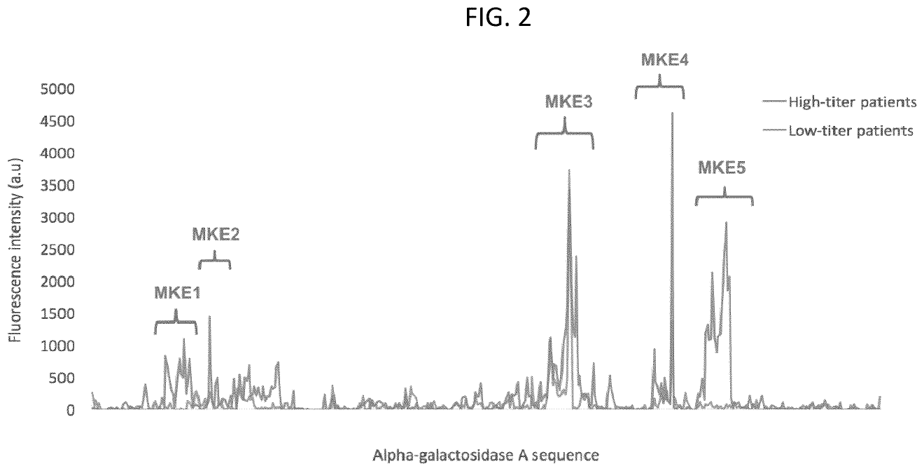

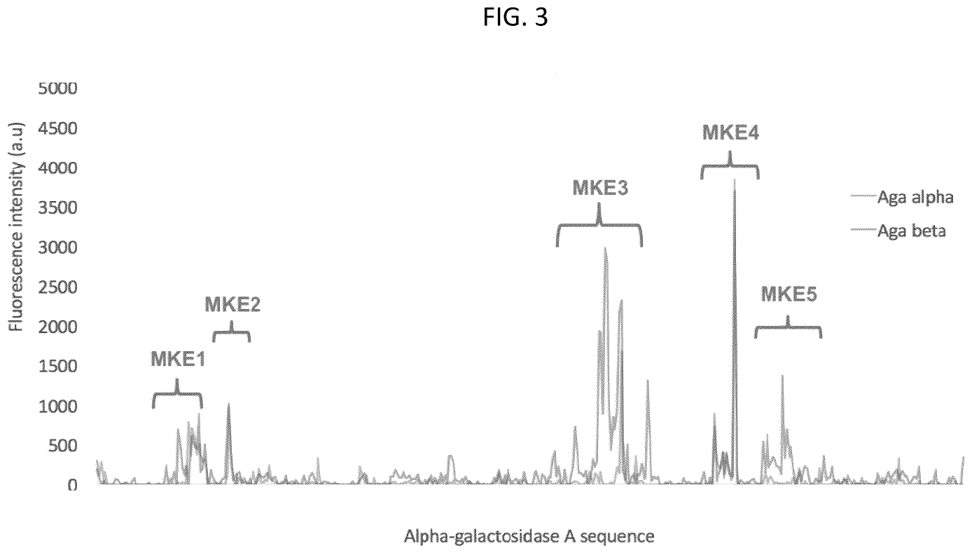

FIG. 2 shows epitope mapping analysis performed on the sera of 17 high-titer and 20 low-titer FD patients. 5 reactive epitopes of α-gal A were identified in high-titer patients, while only a single epitope (MKE3) was present in low-titer patients. FIG. 3 shows patient epitope mapping results that were grouped according to their ERT therapy. Sera from aga β-treated patients contained all 5 of the previously identified epitopes while sera from aga α-treated patients was missing epitope MKE5.

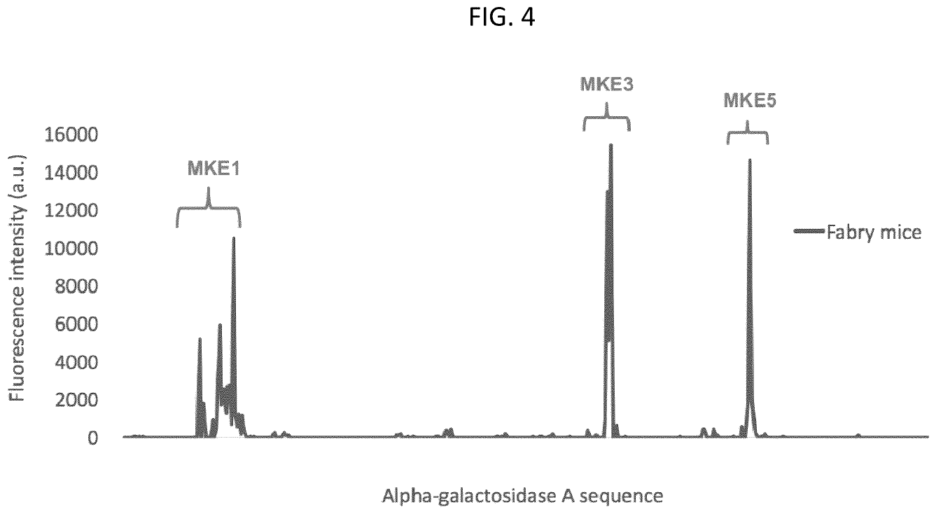

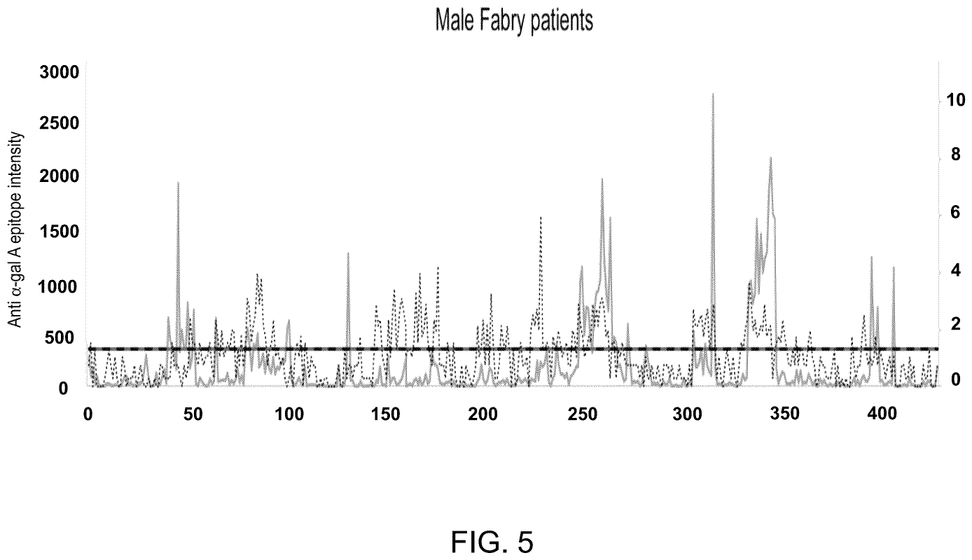

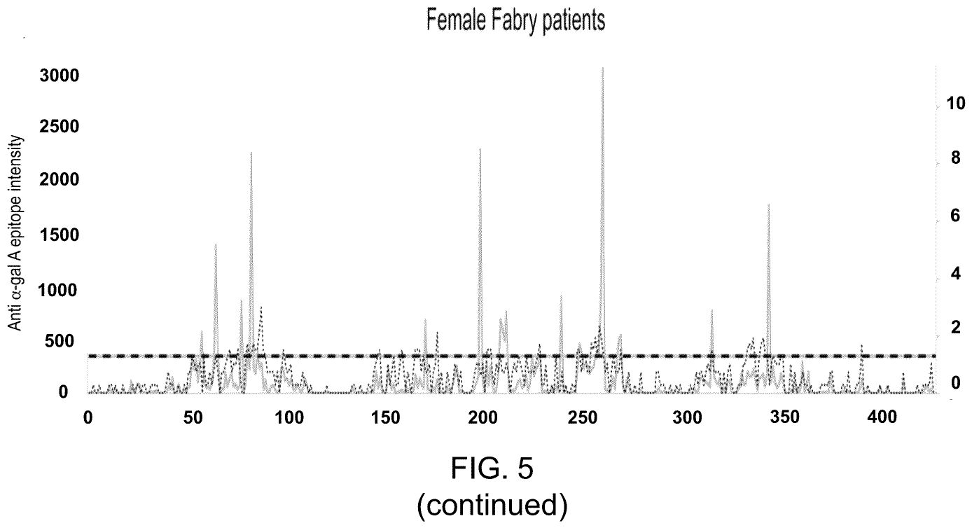

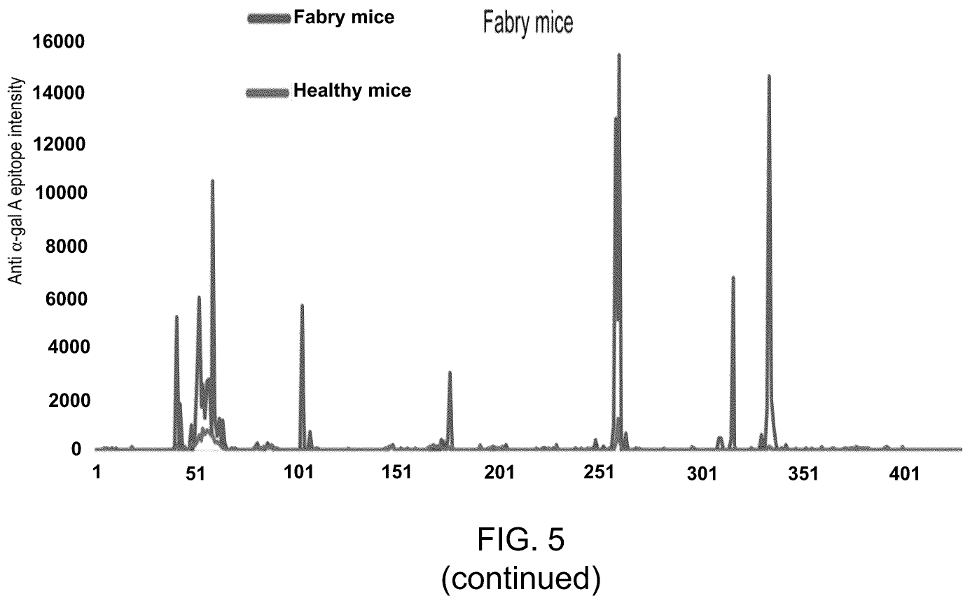

The same analyses were performed in α-gal A immunized Fabry mice to test if mice have an analogous immune reaction. FIG. 4 shows the PEPperMAP which confirmed that the epitope profile detected in mice was similar to that of the high-titer patients (3 of the 5 identified epitopes were present). A comparison of the human and mouse data is shown in FIG. 5. Table 1 lists the immunoreactive α-gal A epitopes identified in male and female Fabry patients as well as Fabry mice. Epitopes that were characterized as strongly immunoreactive are highlighted in bold.

TABLE 1
List of anti-α-gal A immunoreactive epitopes.

| | | | Males | Females | Mouse |
|---|---|---|---|---|---|
| | | Posi- | Re- | Re- | Re- |
| | Epitope | tion | sponse* | sponse* | sponse |
| A | LHWERF | 45-50 | 3.18 | — | YES |
| | (SEQ ID | | | | |
| | NO: 2) | | | | |
| B | DCQEEPDSCI | 55-64 | 6.34 | — | YES |
| | (SEQ ID | | | | |
| | NO: 3) | | | | |
| C | FMEMAELMVSEQ | 69-79 | 1.69 | 1.79 | no |
| | (SEQ ID | | | | |
| | NO: 4) | | | | |
| D | KDAGYEY | 82-88 | 5.32 | 21.18 | no |
| | (SEQ ID | | | | |
| | NO: 5) | | | | |
| E | DDCWMA | 92-97 | 2.91 | 2.2 | no |
| | (SEQ ID | | | | |
| | NO: 6) | | | | |
| F | EGRLQADPQRF | 103-113 | 1.76 | — | no |
| | (SEQ ID | | | | |
| | NO: 7) | | | | |
| G | TCAGF | 141-145 | 1.84 | — | no |
| | (SEQ ID | | | | |
| | NO: 8) | | | | |
| H | DIDAQTF | 153-159 | 2.81 | 2.31 | no |
| | (SEQ ID | | | | |
| | NO: 9) | | | | |
| I | DLLKFDGC | 165-172 | 1.09 | 6.49 | YES |
| | (SEQ ID | | | | |
| | NO: 10) | | | | |
| J | ENLAD | 178-182 | 2.08 | 1.23 | no |
| | (SEQ ID | | | | |
| | NO: 11) | | | | |
| K | IVYSCEW | 198-204 | 2.03 | 21.5 | no |
| | (SEQ ID | | | | |
| | NO: 12) | | | | |
| L | PLYMWPFQ | 205-212 | 0.73 | — | no |
| | (SEQ ID | | | | |
| | NO: 13) | | | | |
| M | YCNHW | 222-226 | 2.31 | 3.41 | no |
| | (SEQ ID | | | | |
| | NO: 14) | | | | |
| N | SWKSI | 235-239 | 3.86 | - | no |
| | (SEQ ID | | | | |
| | NO: 15) | | | | |
| O | LDWTSFNQER | 243-252 | 11.07 | 3.75 | no |
| | (SEQ ID | | | | |
| | NO: 16) | | | | |
| P | IVDVA | 253-257 | 19.18 | 8.81 | YES |
| | (SEQ ID | | | | |
| | NO: 17) | | | | |
| Q | NDPDML | 263-268 | 15.56 | 28.61 | no |
| | (SEQ ID | | | | |
| | NO: 18) | | | | |
| R | ALLQD | 309-313 | 56.96 | 7.26 | no |
| | (SEQ ID | | | | |
| | NO: 19) | | | | |
| S | QLRQGDNF | 330-337 | 9.77 | 2.15 | YES |
| | (SEQ ID | | | | |
| | NO: 20) | | | | |
| T | EVWERPLSG | 338-346 | 20.33 | 16.62 | no |
| | (SEQ ID | | | | |
| | NO: 21) | | | | |
| U | WAVAMIN | 349-355 | 1.73 | 1.77 | no |
| | (SEQ ID | | | | |
| | NO: 22) | | | | |
| V | EIGGPRSY | 358-365 | 1.48 | 6.77 | no |
| | (SEQ ID | | | | |
| | NO: 23) | | | | |

*Intensity per epitope/global mean in arbitrary units.

### Example 2: Modification of α-Gal a to Reduce Immunogenicity

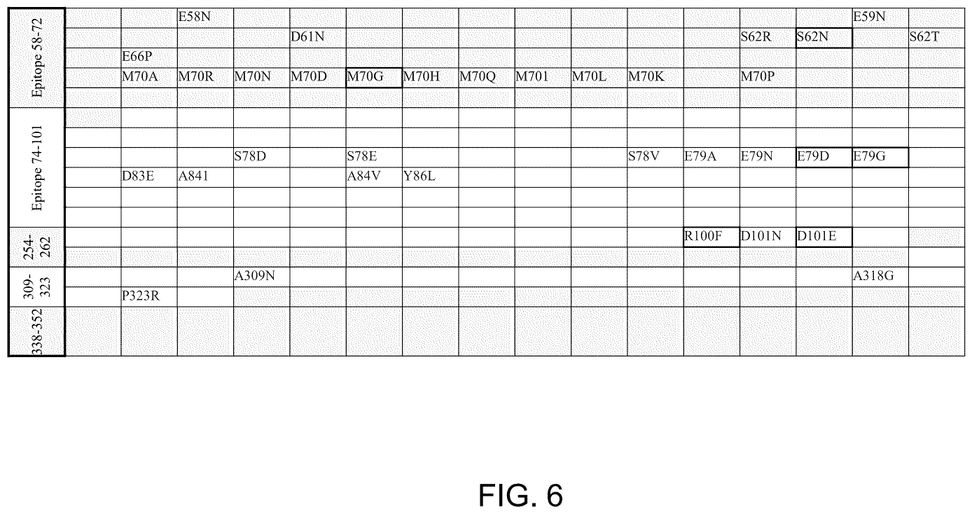

Novel α-gal A variants were selected using for further testing using in silico analyses. Specifically, the selected mutations were predicted to maintain enzyme functionality (software used: PolyPhen-2) while reducing immunogenicity (databases used: CTLpred, Immunomedicine group, MHCBP, IEDB.org, SVRMHC, BcePred, SVMTrip, PREDIVAC, EpiJen). Importantly, amino acid mutations that are known to be associated with Fabry disease were excluded from the list of candidate mutations (databases used: HGMD, Fabrydatabase, Fabry_CEP). The 35 selected α-gal A mutations are shown in Table 2 and in FIG. 6. Notably, these mutations are found in three of the five reactive epitopes identified in Example 1.

TABLE 2
List of mutations that do not affect the functionality of the protein
and make it less immunogenic (according the prediction software).

| | | | | | | |
|---|---|---|---|---|---|---|
| 58/72 | E58N | E59N | D61N | S62R | S62N | S62T |
| | E66P | M70A | M70R | M70N | M70D | M70G |
| | M70H | M70Q | M70I | M70L | M70K | M70P |
| 74-101 | S78D | S78E | S78V | E79A | E79N | E79D |
| | E79G | D83E | A84I | A84V | Y86L | R100F |
| | D101N | D101E | | | | |
| 309-323 | A309N | A318G | P323R | | | |

### Example 3: In Vitro Testing of Selected α-Gal a Variants

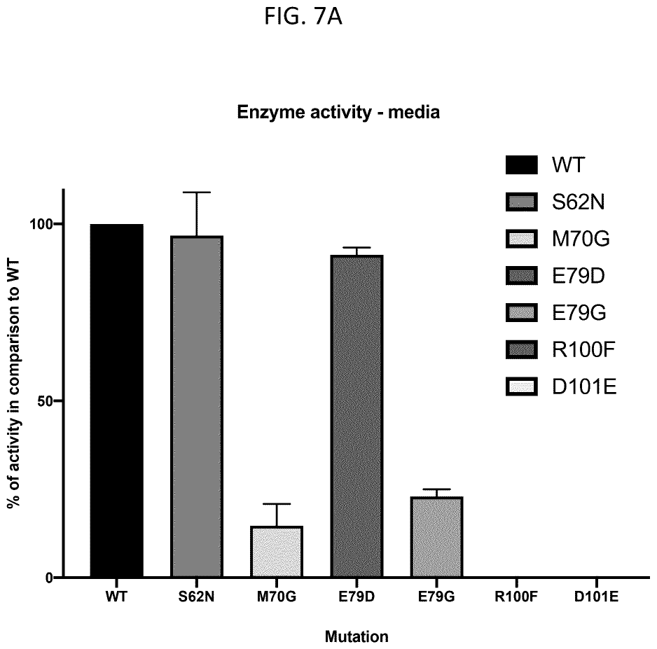

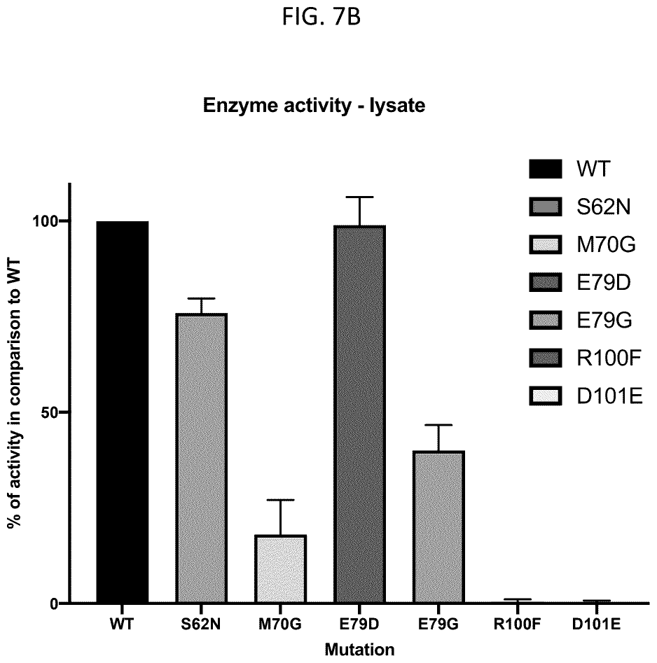

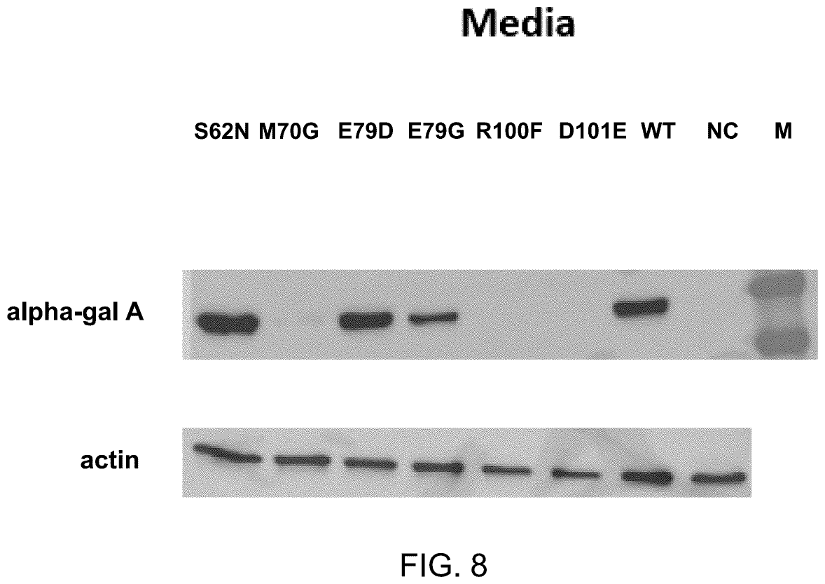

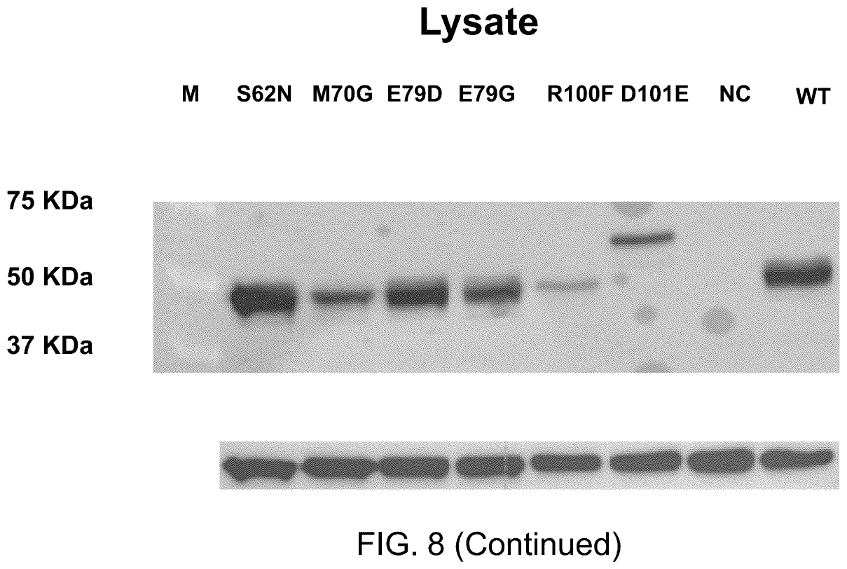

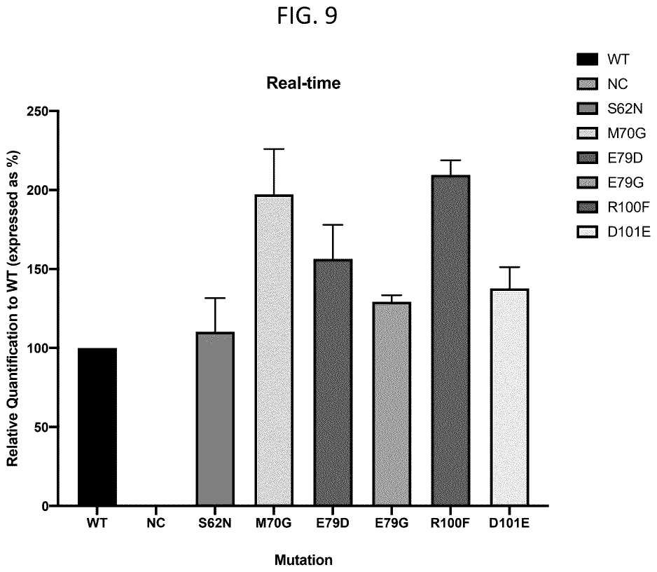

Six mutations (S62N, M70G, E79D, E79G, R100F, and D101E) were selected for a preliminary in vitro analysis. The mutations were generated using site-directed mutagenesis into pcDNA3.4 containing the coding sequence for amino acids 1-429 of human α-gal A, verified by sequencing, and transfected by standard Lipofectamine techniques into HEK 293T cells for expression in triplicate. Enzyme activity assays were performed using both the cell culture media (FIG. 7A) and the lysate of the transfected cells (FIG. 7B). Activity was determined by a fluorometric assay which measured the conversion of 4-methylumbelliferyl-α-D-galactopyranoside substrate. Expression of the mutant proteins was confirmed using western blot analysis in the presence of WPRE (FIG. 8) and quantitative reverse transcription PCR (FIG. 9). The results of these studies demonstrate that two of tested variants (S62N and E79D) were expressed well at the protein level and exhibited enzyme activity levels that were comparable to the wild-type enzyme.

The remaining candidate mutations (listed in Table 2) will be analyzed in future studies. The immunogenicity of any promising α-gal A variants will then be evaluated in vivo using a Fabry mouse model.

Each publication, patent, and patent publication cited in this disclosure is incorporated in reference herein in its entirety. The present invention is not intended to be limited to the foregoing examples but encompasses all such modifications and variations as come within the scope of the appended claims.

1. A less immunogenic mutant α-galactosidase A protein (α-gal) comprising one or more amino acid changes in one or more antigenic epitopes, wherein the one or more antigenic epitopes are selected from the group consisting of
(a)
(SEQ ID NO: 2; position 45-50 of α-gal),
LHWERF
(b)
(SEQ ID NO: 3; position 55-64 of α-gal),
DCQEEPDSCI
(c)
(SEQ ID NO: 4; position 69-79 of α-gal),
FMEMAELMVSEQ
(d)
(SEQ ID NO: 5; position 82-88 of α-gal),
KDAGYEY
(e)
(SEQ ID NO: 6; position 92-97 of α-gal),
DDCWMA
(f)
(SEQ ID NO: 7; position 103-113 of α-gal),
EGRLQADPQRF
(g)
(SEQ ID NO: 8; position 141-145 of α-gal),
TCAGF
(h)
(SEQ ID NO: 9; position 153-159 of α-gal),
DIDAQTF
(i)
(SEQ ID NO: 10; position 165-172 of α-gal),
DLLKFDGC
(j)
(SEQ ID NO: 11; position 178-182 of α-gal),
ENLAD
(k)
(SEQ ID NO: 12; position 198-204 of α-gal),
IVYSCEW
(l)
(SEQ ID NO: 13; position 205-212 of α-gal),
PLYMWPFQ
and
(m)
(SEQ ID NO: 14; position 222-226 of α-gal),
YCNHW
(n)
(SEQ ID NO: 15; position 235-239 of α-gal),
SWKSI
(o)
(SEQ ID NO: 16; position 243-252 of α-gal),
LDWTSFNQER
(p)
(SEQ ID NO: 17; position 253-257 of α-gal),
IVDVA
(q)
(SEQ ID NO: 18; position 263-268 of α-gal),
NDPDML
(r)
(SEQ ID NO: 19; position 309-313 of α-gal),
ALLQD
(s)
(SEQ ID NO: 20; position 330-337 of α-gal),
QLRQGDNF
(t)
(SEQ ID NO: 21; position 338-346 of α-gal),
EVWERPLSG
(u)
(SEQ ID NO: 22; position 349-355 of α-gal),
WAVAMIN
AND
(v)
(SEQ ID NO: 23; position 358-365 of α-gal),
EIGGPRSY
wherein the mutant α-gal retains enzymatic function and is less immunogenic than the non-mutated α-gal.

2. The less immunogenic mutant α-gal of claim 1, wherein the mutant α-gal comprises changes in two or more antigenic epitopes selected from the group consisting of (a)-(v), wherein the mutant α-gal retains enzymatic function and is less immunogenic than the non-mutated α-gal.

3. The less immunogenic mutant α-gal of claim 1, wherein the mutant α-gal comprises changes in three or more antigenic epitopes selected from the group consisting of (a)-(v), wherein the mutant α-gal retains enzymatic function and is less immunogenic than the non-mutated α-gal.

4. The less immunogenic mutant α-gal of claim 1, wherein the mutant α-gal comprises amino acid change at one or more positions within the α-gal protein, wherein the one or more positions is selected from the group consisting of 58, 59, 61, 62, 66, 70, 78, 79, 83, 84, 86, 100, 101, 309, 318, and 323, wherein the amino acid change at one or more of these positions retains enzymatic function and reduces the immune reaction to exogenous α-gal in a subject.

5. The less immunogenic mutant α-gal of claim 1, wherein the mutant α-gal comprises one or more amino acid change selected from the group consisting of E58N, E59N, D61N, S62R, S62N, S62T, E66P, M70A, M70R, M70N, M70D, M70G, M70H, M70Q, M70I, M70L, M70K, M70P, S78D, S78E, S78V, E79A, E79N, E79D, E79G, D83E, A84I, A84V, Y86L, R100F, D101N, D101E, A309N, A318G, and P323R.

6. The mutant α-gal of claim 1, wherein the one or more amino acid changes are within the sequence of α-gal of SEQ ID NO:1.

7. An expression construct comprising a nucleic acid sequence encoding the mutant α-gal of claim 1.

8. The expression construct of claim 7, wherein the expression construct comprises a heterologous promoter and is capable of expressing the mutant α-gal in a cell.

9. The expression construct of claim 7, wherein the construct is a viral construct.

10. A virus comprising the expression construct of claim 9.

11. A cell able to express the mutant α-gal of claim 1.

12. The cell of claim 11, wherein the cell comprises the expression construct of claim 7.

13. A method of producing a mutant α-gal, the method comprising:
(a) expressing the mutant α-gal of claim 1 in a cell; and
(b) isolating and purifying the mutant α-gal protein from the cell, wherein the mutant α-gal has enzymatic activity.

14. The method of claim 13, wherein the cell is a tissue culture cell.

15. The method of claim 13, wherein the cell comprises an expression construct able to express the mutant α-gal.

16. A method of treating Fabry disease in a subject, the method comprising the steps of:
(a) administering a therapeutically effective amount of the mutant α-gal of claim 1 to treat Fabry disease and reduce one or more symptoms of the lysosomal storage disorder.

17. The method of claim 16, wherein the mutant α-gal is administered by intravenous transfusion.

18. The method of claim 16, wherein the mutant α-gal is administered by gene therapy.


SEQUENCE LISTING (23 sequences)

SEQ ID NO: 1 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 2 - protein, 6 aa, Homo sapiens
LHWERF

SEQ ID NO: 3 - protein, 10 aa, Homo sapiens
DCQEEPDSCI

SEQ ID NO: 4 - protein, 12 aa, Homo sapiens
FMEMAELMVSEQ

SEQ ID NO: 5 - protein, 7 aa, Homo sapiens
KDAGYEY

SEQ ID NO: 6 - protein, 6 aa, Homo sapiens
DDCWMA

SEQ ID NO: 7 - protein, 11 aa, Homo sapiens
EGRLQADPQRF

SEQ ID NO: 8 - protein, 5 aa, Homo sapiens
TCAGF

SEQ ID NO: 9 - protein, 7 aa, Homo sapiens
DIDAQTF

SEQ ID NO: 10 - protein, 8 aa, Homo sapiens
DLLKFDGC

SEQ ID NO: 11 - protein, 5 aa, Homo sapiens
ENLAD

SEQ ID NO: 12 - protein, 7 aa, Homo sapiens
IVYSCEW

SEQ ID NO: 13 - protein, 8 aa, Homo sapiens
PLYMWPFQ

SEQ ID NO: 14 - protein, 5 aa, Homo sapiens
YCNHW

SEQ ID NO: 15 - protein, 5 aa, Homo sapiens
SWKSI

SEQ ID NO: 16 - protein, 10 aa, Homo sapiens
LDWTSFNQER

SEQ ID NO: 17 - protein, 5 aa, Homo sapiens
IVDVA

SEQ ID NO: 18 - protein, 6 aa, Homo sapiens
NDPDML

SEQ ID NO: 19 - protein, 5 aa, Homo sapiens
ALLQD

SEQ ID NO: 20 - protein, 8 aa, Homo sapiens
QLRQGDNF

SEQ ID NO: 21 - protein, 9 aa, Homo sapiens
EVWERPLSG

SEQ ID NO: 22 - protein, 7 aa, Homo sapiens
WAVAMIN

SEQ ID NO: 23 - protein, 8 aa, Homo sapiens
EIGGPRSY